(19) United States
(12) Patent Application Publication
(10) Pub. No.: US 2024/0325397 A1
(43) Pub. Date: Oct. 3, 2024

(54) USE OF COMBINATION THERAPY FOR TREATING CANCER

(71) Applicant: Recurium IP Holdings, LLC, San Diego, CA (US)

(72) Inventors: Ahmed Abdi Samatar, West Windsor, NJ (US); Fernando Donate, Santa Monica, CA (US); Jiali Li, San Diego, CA (US); Jianhui Ma, San Diego, CA (US); Kevin Duane Bunker, Escondido, CA (US); Peter Qinhua Huang, San Diego, CA (US)

(21) Appl. No.: 18/743,564

(22) Filed: Jun. 14, 2024

Related U.S. Application Data
(63) Continuation of application No. PCT/US2022/081592, filed on Dec. 14, 2022.
(60) Provisional application No. 63/265,441, filed on Dec. 15, 2021.

(51) Int. Cl. A61K 31/519; A61K 39/00; A61K 45/06; A61P 35/00

(52) U.S. Cl. CPC A61K 31/519; A61K 45/06; A61P 35/00; A61K 2039/505

(57) ABSTRACT

Disclosed herein are combinations of compounds for treating a disease or condition, such as cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/265,441, filed Dec. 15, 2021, which is incorporated by reference in its entirety. The present application is a continuation of PCT Application No. PCT/US2022/081592, filed Dec. 14, 2022, which claims priority to U.S. Provisional Application No. 63/265,441, filed Dec. 15, 2021, each of which is hereby incorporated by reference in their entireties.

### FIELD

The present application relates to the fields of chemistry, biochemistry and medicine. More particularly, disclosed herein are combination therapies, and methods of treating diseases and/or conditions with combination therapies described herein.

### DESCRIPTION

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2019, roughly 1.8 million people will be diagnosed with cancer, and an estimated 606,880 people will die of cancer in the United States. Thus, there still exists a need for effective cancer treatments.

### SUMMARY

Some embodiments described herein provide, among other things, combination therapies comprising a WEE1 inhibitor (e.g., Compound A) and a CDK4/6 inhibitor or a HER-2 inhibitor for use in treating a disease or condition (e.g., cancer). In some embodiments, the HER-2 inhibitor is selected from a HER-2 small molecule inhibitor, a HER-2 antibody, a HER-2 antibody-drug conjugate and a HER2 bispecific antibody, or a pharmaceutically acceptable salt of any of the foregoing. In some embodiments, the HER-2 inhibitor is selected from a HER-2 antibody, a HER-2 antibody-drug conjugate and a HER2 bispecific antibody, or a pharmaceutically acceptable salt of any of the foregoing.

In one aspect, some embodiments provide a method of treating cancer comprising administering to a subject an effective amount of Compound (A) and an effective amount of Compound (B), or a pharmaceutically acceptable salt of any of the foregoing, wherein:

Compound (A) is

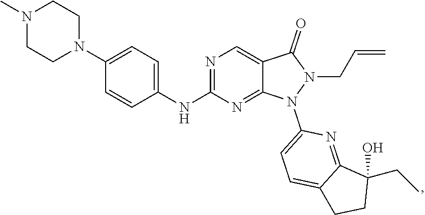 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; and Compound (B) is selected from the group consisting of a CDK4/6 inhibitor, a HER-2 small molecule inhibitor, a HER-2 antibody, a HER-2 antibody-drug conjugate and a HER2 bispecific antibody, or a pharmaceutically acceptable salt of any of the foregoing.

In one aspect, some embodiments provide a method of treating cancer comprising administering to a subject an effective amount of Compound (A) and an effective amount of Compound (B), or a pharmaceutically acceptable salt of any of the foregoing, wherein:

Compound (A) is

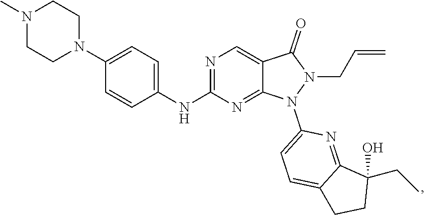 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; and Compound (B) is selected from the group consisting of a HER-2 small molecule inhibitor, a HER-2 antibody, a HER-2 antibody-drug conjugate and a HER2 bispecific antibody, or a pharmaceutically acceptable salt of any of the foregoing.

In some embodiments, Compound (B) is a HER-2 small molecule inhibitor, or a pharmaceutically acceptable salt thereof. In some embodiments, Compound (B) is a HER-2 antibody. In some embodiments, Compound (B) is a HER-2 antibody-drug conjugate, or a pharmaceutically acceptable salt thereof. In some embodiments, Compound (B) is a HER-2 bispecific antibody, or a pharmaceutically acceptable salt thereof.

In one aspect, some embodiments provide a method of treating cancer comprising administering to a subject an effective amount of Compound (A) and an effective amount of Compound (B), or a pharmaceutically acceptable salt of any of the foregoing, wherein:

Compound (A) is

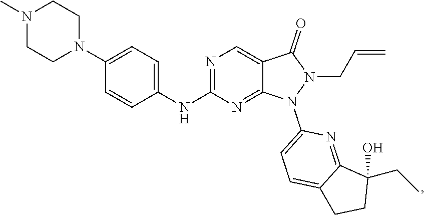 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; and Compound (B) is a CDK4/6 inhibitor, or a pharmaceutically acceptable salt thereof.

In one aspect, some embodiments provide a method of treating triple negative breast cancer comprising administering to a subject an effective amount of Compound (A) and an effective amount of Compound (B), or a pharmaceutically acceptable salt of any of the foregoing, wherein:

Compound (A) is

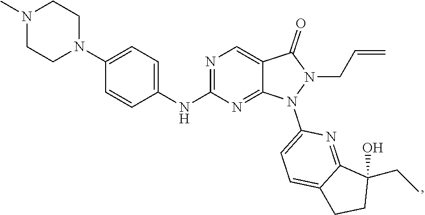 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; and

Compound (B) is fam-trastuzumab-deruxtecan-nxki (DS8201a).

Some embodiments described herein relate to a combination of compounds that can include an effective amount of Compound (A), or a pharmaceutically acceptable salt thereof, and an effective amount of Compound (B), or a pharmaceutically acceptable salt of any of the foregoing.

Some embodiments described herein relate to the use of a combination of compounds for treating a disease or condition, wherein the combination includes an effective amount of Compound (A), or a pharmaceutically acceptable salt thereof, and an effective amount of Compound (B), or a pharmaceutically acceptable salt of any of the foregoing. Other embodiments described herein relate to the use of a combination of compounds in the manufacture of a medicament for treating a disease or condition, wherein the combination includes an effective amount of Compound (A), or a pharmaceutically acceptable salt thereof, and an effective amount of Compound (B), or a pharmaceutically acceptable salt of any of the foregoing.

In some embodiments, the disease or condition can be a cancer described herein.

In one aspect, some embodiments provide a use of a combination of compounds for treating a disease or condition, wherein the combination includes an effective amount of Compound (A) and an effective amount of Compound (B), or a pharmaceutically acceptable salt of any of the foregoing, wherein:

Compound (A) is

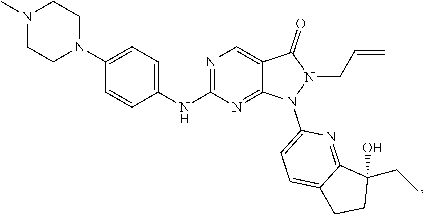 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; and

Compound (B) is selected from the group consisting of a CDK4/6 inhibitor, a HER-2 small molecule inhibitor, a HER-2 antibody, a HER-2 antibody-drug conjugate and a HER2 bispecific antibody, or a pharmaceutically acceptable salt of any of the foregoing;

wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002 and MM-D37K, or a pharmaceutically acceptable salt of any of the foregoing;

wherein the HER-2 antibody is selected from the group consisting of trastuzumab, trastuzumab-dkst, pertuzumab and ZW25, or a pharmaceutically acceptable salt of any of the foregoing;

wherein the HER-2 antibody-drug conjugate is selected from the group consisting of Ado-trastuzumab emtansine (T-DM1), ARX788, ALT-P7, Enhertu® (fam-trastuzumab-deruxtecan-nxki, DS8201a), MEDI4276, MM302, PF-06804103, SYD985, XMT-1522, ZW49, MRG002, GQ1001, A166, RC48-ADC, BDC-1001, and FS-1502, or a pharmaceutically acceptable salt of any of the foregoing; and

wherein the HER2 bispecific antibody is selected from the group consisting of margetuximab, ertumaxomab, HER2Bi-aATC, MM-111, MCLA-128, BTRC4017A, GBR-1302 and PRS-343, or a pharmaceutically acceptable salt of any of the foregoing.

Exemplary HER-2 antibody-drug conjugates are described in Ferraro et al., Implementing antibody-drug conjugates (ADCs) in HER2-positive breast cancer: state of the art and future directions. Breast Cancer Res (2021) 23(1):84. (https://doi.org/10.1186/s13058-021-01459-y), which is hereby incorporated by reference in its entirety.

In some embodiments, Compound (B) is a HER-2 small molecule inhibitor, or a pharmaceutically acceptable salt thereof. In some embodiments, Compound (B) is a HER-2 small molecule inhibitor selected from tucatinib, lapatinib and neratinib, or a pharmaceutically acceptable salt of any of the foregoing. In some embodiments, Compound (B) is tucatinib, or a pharmaceutically acceptable salt thereof. In some embodiments, Compound (B) is lapatinib, or a pharmaceutically acceptable salt thereof. In some embodiments, Compound (B) is neratinib, or a pharmaceutically acceptable salt thereof. In some embodiments, a HER-2 small molecule inhibitor is administered in combination with a HER-2 antibody (e.g., trastuzumab).

In some embodiments, Compound (B) is a CDK4/6 inhibitor, or a pharmaceutically acceptable salt thereof. In some embodiments, Compound (B) is a CDK4/6 inhibitor, or a pharmaceutically acceptable salt thereof selected from FIG. 1.

In some embodiments, the CDK4/6 inhibitor is palbociclib.

In some embodiments, the CDK4/6 inhibitor is abemaciclib.

In some embodiments, the CDK4/6 inhibitor is ribociclib.

In some embodiments, the CDK4/6 inhibitor is trilaciclib.

In some embodiments, Compound (B) is an HER-2 antibody, or a pharmaceutically acceptable salt thereof.

In some embodiments, the HER-2 antibody is trastuzumab.

In some embodiments, Compound (B) is a HER-2 antibody-drug conjugate, or a pharmaceutically acceptable salt thereof. In some embodiments, Compound (B) is a trastuzumab antibody-drug conjugate, or a pharmaceutically acceptable salt thereof. In some embodiments, Compound (B) is fam-trastuzumab-deruxtecan-nxki (DS8201a).

In some embodiments, Compound (B) is a HER2 bispecific antibody, or a pharmaceutically acceptable salt thereof.

In some embodiments, Compound (B) is selected from FIG. 2.

In some embodiments, Compound (B) is a HER-2 small molecule inhibitor, or a pharmaceutically acceptable salt thereof. In some embodiments, Compound (B) is a HER-2 small molecule inhibitor selected from tucatinib, lapatinib and neratinib, or a pharmaceutically acceptable salt of any of the foregoing. In some embodiments, a HER-2 small molecule inhibitor is administered in combination with a HER-2 antibody (e.g., trastuzumab), or a pharmaceutically acceptable salt thereof.

In some embodiments, the disease or condition is a breast cancer.

In some embodiments, the disease or condition is selected from the group consisting of triple-negative breast cancer (TNBC) and estrogen receptor positive (ER+) breast cancer. In some embodiments, the breast cancer is ER positive (ER+) breast cancer. In some embodiments, the breast cancer is ER positive, HER2-negative (ER+/HER2−) breast cancer. In some embodiments, the breast cancer is triple-negative breast cancer (TNBC).

In some embodiments, the breast cancer is classified by HER2 status. In some embodiments, the breast cancer is HER2-positive (HER2+) breast cancer. In some embodiments, the breast cancer is HER2-low breast cancer. In some embodiments, the breast cancer is classified as HER2 negative (HER2−) breast cancer.

In some embodiments, the disease or condition is lung cancer, gastric cancer or gastroesophageal junction adenocarcinoma.

In one aspect, some embodiments provide use of an effective amount of Compound (A), or a pharmaceutically acceptable salt of any of the foregoing, in the preparation or manufacture of a medicament for treating ER+ breast cancer, wherein Compound (A) is

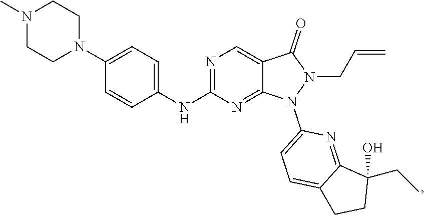 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof.

In some embodiments, the breast cancer does not include any ER point mutations.

In some embodiments, the breast cancer has at least one point mutation within the Estrogen Receptor 1 (ESR1) that encodes Estrogen receptor alpha (ERα), wherein the mutation is selected from the group consisting of: K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M4371, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H.

In some embodiments, the breast cancer is ER positive breast cancer.

In some embodiments, the breast cancer is ER positive/HER2-negative breast cancer.

In some embodiments, the breast cancer is local breast cancer.

In some embodiments, the breast cancer is metastatic breast cancer.

In some embodiments, the breast cancer is recurrent breast cancer.

In some embodiments, the breast cancer has been previously treated with an endocrine therapy. In some embodiments, the treatment was with a selective ER modulator (SERM). In some embodiments, the selective ER modulator is selected from the group consisting of tamoxifen, raloxifene, ospemifene, bazedoxifene, toremifene and lasofoxifene, or a pharmaceutically acceptable salt of any of the foregoing.

In some embodiments, the previous treatment was with a selective ER degrader (SERD). In some embodiments, the selective ER degrader is selected from the group consisting of fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R) -6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl) -6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250 and LY3484356, or a pharmaceutically acceptable salt of any of the foregoing.

In some embodiments, the previous treatment was with an aromatase inhibitor. In some embodiments, the aromatase inhibitor is a steroidal aromatase inhibitor.

In some embodiments, the steroidal aromatase inhibitor is selected from the group consisting of exemestane and testolactone, or a pharmaceutically acceptable salt of any of the foregoing.

In some embodiments, the aromatase inhibitor is a non-steroidal aromatase inhibitor. In some embodiments, the non-steroidal aromatase inhibitor is selected from the group consisting of anastazole and letrazole, or a pharmaceutically acceptable salt of any of the foregoing.

In some embodiments, the breast cancer has not been previously treated.

In some embodiments, the breast cancer is present in a woman. In some embodiments, the woman is a premenopausal woman. In some embodiments, the woman is a perimenopausal woman. In some embodiments, the woman is a menopausal woman. In some embodiments, the breast cancer is present in a postmenopausal woman.

In some embodiments, the breast cancer is present in a man.

In some embodiments, the breast cancer is present in a human subject. In some embodiments, the breast cancer is present in a subject that has a serum estradiol level in the range of >15 pg/mL to 350 pg/mL.

In some embodiments, the breast cancer is present in a subject that has a serum estradiol level in the range of >15 pg/mL to 300 pg/mL, >20 pg/mL to 350 pg/mL, >25 pg/mL to 350 pg/mL, >30 pg/mL to 350 pg/mL, >35 pg/mL to 350 pg/mL, >40 pg/mL to 350 pg/mL, >45 pg/mL to 350 pg/mL, >50 pg/mL to 350 pg/mL, >55 pg/mL to 350 pg/mL, >60 pg/mL to 350 pg/mL, >65 pg/mL to 350 pg/mL, >70 pg/mL to 350 pg/mL, >75 pg/mL to 350 pg/mL, >80 pg/mL to 350 pg/mL, >85 pg/mL to 350 pg/mL, >90 pg/mL to 350 pg/mL, >95 pg/mL to 350 pg/mL, >100 pg/mL to 350 pg/mL, >125 pg/mL to 350 pg/mL, >150 pg/mL to 350 pg/mL, >200 pg/mL to 350 pg/mL, >250 pg/mL to 350 pg/mL, or >300 pg/mL to 350 pg/mL.

In some embodiments, the breast cancer is present in a subject that has a serum estradiol level ≤15 pg/mL. In some embodiments, the breast cancer is present in a subject that has a serum estradiol level ≤10 pg/mL.

In some embodiments, the breast cancer is present in a subject that has a serum estradiol level ≤20 pg/mL, ≤19 pg/mL, ≤18 pg/mL, ≤17 pg/mL≤16 pg/mL, ≤15 pg/mL≤14 pg/mL≤13 pg/mL, ≤12 pg/mL,≤11 pg/mLor≤10 pg/mL.

In one aspect, some embodiments provide a method of treating cancer comprising administering to a subject an effective amount of Compound (A) and an effective amount of Compound (B), or a pharmaceutically acceptable salt of any of the foregoing, wherein:

Compound (A) is

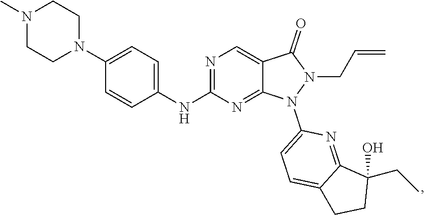 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof; and

Compound (B) is selected from the group consisting of a CDK4/6 inhibitor, a HER-2 small molecule inhibitor, a HER-2 antibody, a HER-2 antibody-drug conjugate and a HER2 bispecific antibody, or a pharmaceutically acceptable salt of any of the foregoing;

wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002 and MM-D37K, or a pharmaceutically acceptable salt of any of the foregoing;

wherein the HER-2 antibody is selected from the group consisting of trastuzumab, trastuzumab-dkst, pertuzumab and ZW25, or a pharmaceutically acceptable salt of any of the foregoing;

wherein the HER-2 antibody-drug conjugate is selected from the group consisting of Ado-trastuzumab emtansine (T-DM1), ARX788, ALT-P7, DS8201a, MEDI4276, MM302, PF-06804103, SYD985, XMT-1522, ZW49, MRG002, GQ1001, A166, RC48-ADC, BDC-1001, and FS-1502, or a pharmaceutically acceptable salt of any of the foregoing; and

wherein the HER2 bispecific antibody is selected from the group consisting of margetuximab, ertumaxomab, HER2Bi-aATC, MM-111, MCLA-128, BTRC4017A, GBR-1302 and PRS-343, or a pharmaceutically acceptable salt of any of the foregoing.

In some embodiments, Compound (B) is a HER-2 small molecule inhibitor, or a pharmaceutically acceptable salt thereof. In some embodiments, Compound (B) is a HER-2 small molecule inhibitor selected from tucatinib, lapatinib and neratinib, or a pharmaceutically acceptable salt of any of the foregoing. In some embodiments, a HER-2 small molecule inhibitor is administered in combination with a HER-2 antibody (e.g., trastuzumab), or a pharmaceutically acceptable salt thereof.

### DRAWINGS

FIG. 1 provides examples of CDK4/6 inhibitors.

FIG. 2 provides examples of HER-2 antibodies, HER-2 antibody-drug conjugates and a HER2 bispecific antibodies.

FIG. 3 shows the results of an in vivo study of Compound (A), or a pharmaceutically acceptable salt thereof, in a MCF-7 xenograft model.

FIG. 4 shows the results of an in vivo study of Compound (A), or a pharmaceutically acceptable salt thereof, or Tamoxifen in a ZR-75-1 tamoxifen resistant tumor model.

FIG. 5 shows the results of an in vivo study of Compound (A), or a pharmaceutically acceptable salt thereof, in human HCC1428 breast cancer xenograft tumors.

FIG. 6 shows the results of an in vivo study of Compound (A), or a pharmaceutically acceptable salt thereof, as mono-therapy or in combination with a CDK4/6 inhibitor, Palbociclib, in a MCF-7 xenograft model.

FIG. 7 shows the results of an in vivo study of Compound (A), or a pharmaceutically acceptable salt thereof, as mono-therapy or in combination with a CDK4/6 inhibitor, palbociclib, in a MCF-7 xenograft model.

FIG. 8 provides the tumor volume changes in mice treated with vehicle, Compound (A), trastuzumab or combination of Compound (A) with trastuzumab.

FIG. 9 provides the tumor volume changes in mice treated with vehicle, Compound (A), trastuzumab or combination of Compound (A) with trastuzumab.

FIG. 10A and 10B provide changes in tumor volume and body weight, respectively, in palbociclib-resistant breast cancer patient derived xenograft (PDX) model (CTG-1207) mice treated with vehicle, Compound (A), palbociclib or combination of Compound (A) with palbociclib.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic, or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris (hydroxymethyl) methylamine, C₁-C₇ alkylamine, cyclohexylamine, triethanolamine, ethylenediamine, and salts with amino acids such as arginine and lysine. For compounds (A) and (B), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastercomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited clements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to the use of a combination of compounds for treating a disease or condition, wherein the combination can include an effective amount of Compound (A), or a pharmaceutically acceptable salt thereof, and an effective amount of Compound (B), or a pharmaceutically acceptable salt of any of the foregoing, wherein Compound (A) can be a WEE1 inhibitor, or a pharmaceutically acceptable salt thereof; and Compound (B) can be selected from a CDK4/6 inhibitor, a HER-2 antibody, a HER-2 antibody-drug conjugate and a HER2 bispecific antibody (including pharmaceutically acceptable salts of any of the foregoing).

Compound (A), including pharmaceutically acceptable salts thereof, can be

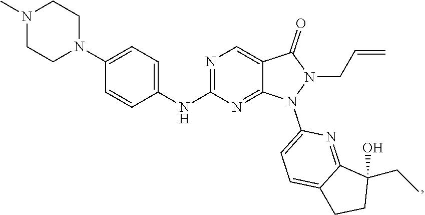 [SMILES: C=CCn1c(=O)c2cnc(Nc3ccc(N4CCN(C)CC4)cc3)nc2n1-c1ccc2c(n1)[C@@](O)(CC)CC2]

including pharmaceutically acceptable salts thereof. In some embodiments, Compound (B), including pharmaceutically acceptable salts thereof, can be a CDK4/6 inhibitor selected from palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002 and MM-D37K (along with pharmaceutically acceptable salts of any of the foregoing). In other embodiments, Compound (B), including pharmaceutically acceptable salts thereof, can be a HER-2 small molecule inhibitor selected from tucatinib, lapatinib and neratinib, or a pharmaceutically acceptable salt of any of the foregoing. In still other embodiments, Compound (B), including pharmaceutically acceptable salts thereof, can be a HER-2 antibody selected from trastuzumab, trastuzumab-dkst, pertuzumab and ZW25 (along with pharmaceutically acceptable salts of any of the foregoing). In yet still other embodiments, Compound (B), including pharmaceutically acceptable salts thereof, can be a HER-2 antibody-drug conjugate selected from Ado-trastuzumab emtansine (T-DM1), ARX788, ALT-P7, DS8201a, MEDI4276, MM302, PF-06804103, SYD985, XMT-1522, ZW49, MRG002, GQ1001, A166, RC48-ADC, BDC-1001, FS-1502 (along with pharmaceutically acceptable salts of any of the foregoing). In some embodiments, Compound (B), including pharmaceutically acceptable salts thereof, can be HER2 bispecific antibody selected from margetuximab, ertumaxomab, HER2Bi-aATC, MM-111, MCLA-128, BTRC4017A, GBR-1302 and PRS-343 (along with pharmaceutically acceptable salts of any of the foregoing).

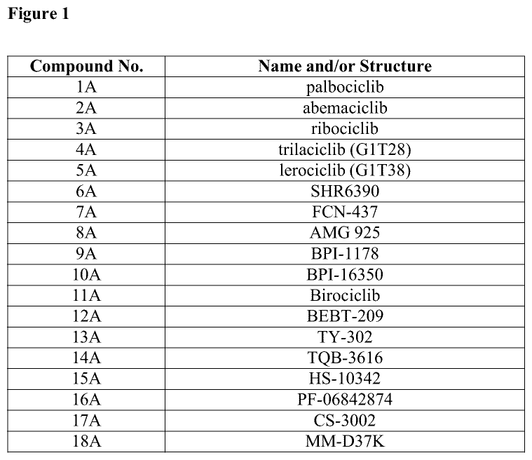

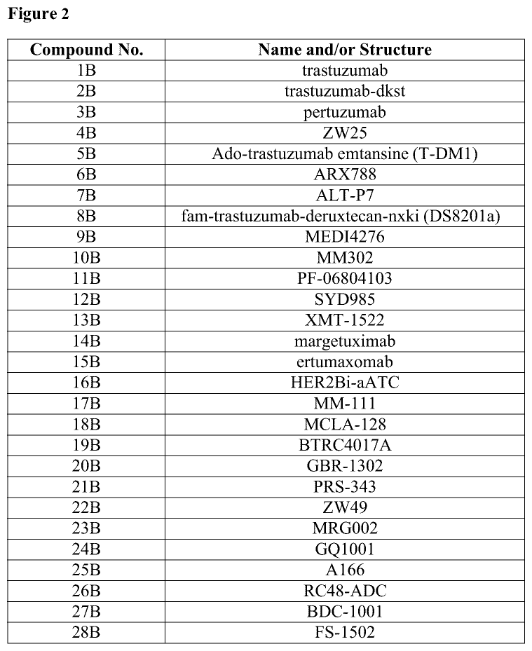

Embodiments of combinations of Compound (A) and Compound (B), including pharmaceutically acceptable salts of any of the foregoing, are provided in Table 1. In Table 1, “A” indicates Compound (A) (including pharmaceutically acceptable salts thereof), the numbers 1A-18A represent a compound as provided in FIG. 1 and numbers 1B-28B represent a compound as provided in FIG. 2, including pharmaceutically acceptable salts thereof. For example, in Table 1, a combination represented by 1A:A corresponds to a combination of palbociclib and

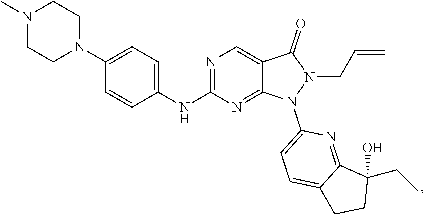 [SMILES: C=CCn1c(=O)c2cnc(Nc3ccc(N4CCN(C)CC4)cc3)nc2n1-c1ccc2c(n1)[C@@](O)(CC)CC2]

including pharmaceutically acceptable salts of any of the foregoing.

TABLE 1

```
Cmpd:Cmpd
  1A:A
  2A:A
  3A:A
  4A:A
  5A:A
  6A:A
  7A:A
  8A:A
  9A:A
10A:A
11A:A
12A:A
13A:A
14A:A
15A:A
16A:A
17A:A
18A:A
  1B:A
  2B:A
  3B:A
  4B:A
  5B:A
  6B:A
  7B:A
  8B:A
  9B:A
10B:A
11B:A
12B:A
13B:A
14B:A
15B:A
16B:A
17B:A
18B:A
19B:A
20B:A
21B:A
22B:A
23B:A
24B:A
25B:A
26B:A
27B:A
28B:A
```

The order of administration of compounds in a combination described herein can vary. In some embodiments, Compound (A), including pharmaceutically acceptable salts thereof, can be administered prior to all of Compound (B), or a pharmaceutically acceptable salt thereof. In other embodiments, Compound (A), including pharmaceutically acceptable salts thereof, can be administered prior to at least one Compound (B), or a pharmaceutically acceptable salt thereof. In still other embodiments, Compound (A), including pharmaceutically acceptable salts thereof, can be administered concomitantly with Compound (B), or a pharmaceutically acceptable salt thereof. In yet still other embodiments, Compound (A), including pharmaceutically acceptable salts thereof, can be administered subsequent to the administration of at least one Compound (B), or a pharmaceutically acceptable salt thereof. In some embodiments, Compound (A), including pharmaceutically acceptable salts thereof, can be administered subsequent to the administration of all Compound (B), or a pharmaceutically acceptable salt thereof.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a combination as described herein of Compound (A), including pharmaceutically acceptable salts thereof, and Compound (B), or pharmaceutically acceptable salts thereof, can decrease the number and/or severity of side effects that can be attributed to a compound described herein, such as Compound (B), or a pharmaceutically acceptable salt thereof.

Using a combination of compounds described herein can results in additive, synergistic or strongly synergistic effect. A combination of compounds described herein can result in an effect that is not antagonistic.

In some embodiments, a combination as described herein of Compound (A), including pharmaceutically acceptable salts thereof, and Compound (B), or pharmaceutically acceptable salts thereof, can result in an additive effect. In some embodiments, a combination as described herein of Compound (A), including pharmaceutically acceptable salts thereof, and Compound (B), or pharmaceutically acceptable salts thereof, can result in a synergistic effect. In some embodiments, a combination as described herein of Compound (A), including pharmaceutically acceptable salts thereof, and Compound (B), or pharmaceutically acceptable salts thereof, can result in a strongly synergistic effect. In some embodiments, a combination as described herein of Compound (A), including pharmaceutically acceptable salts thereof, and Compound (B), or pharmaceutically acceptable salts thereof, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of cach compound is determined individually.

A potential advantage of utilizing a combination as described herein may be a reduction in the required amount(s) of the compound(s) that is effective in treating a disease condition disclosed herein compared to when each compound is administered as a monotherapy. For example, the amount of Compound (B), or a pharmaceutically acceptable salt thereof, used in a combination described herein can be less compared to the amount of Compound (B), or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A), including pharmaceutically acceptable salts thereof, can be provided in a pharmaceutical composition. Likewise, Compound (B), including pharmaceutically acceptable salts thereof, can be provided in a pharmaceutical composition.

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

In some embodiments, Compounds (B), along with pharmaceutically acceptable salts thereof, can be provided in a pharmaceutical composition that includes Compound (A), including pharmaceutically acceptable salts thereof. In other embodiments, Compound (B), along with pharmaceutically acceptable salts thereof, can be administered in a pharmaceutical composition that is separate from a pharmaceutical composition that includes Compound (A), including pharmaceutically acceptable salts thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A), including pharmaceutically acceptable salts thereof, can be administered orally. In some embodiments, Compound (A), including pharmaceutically acceptable salts thereof, can be provided to a subject by the same route of administration as Compound (B), along with pharmaceutically acceptable salts thereof. In other embodiments, Compound (A), including pharmaceutically acceptable salts thereof, can be provided to a subject by a different route of administration as Compound (B), along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds that includes an effective amount of Compound (A), including pharmaceutically acceptable salts thereof, and an effective amount of Compound (B), or a pharmaceutically acceptable salt of any of the foregoing, can be used to treat a disease or condition.

As used herein, a “subject” refers to an animal that is the object of treatment, observation or experiment. “Animal” includes cold-and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

In some embodiments, the disease or condition can be selected from a lung cancer, a gastric cancer and a gastroesophageal junction adenocarcinoma. In some embodiments, the disease or condition can be a breast cancer. Various types of breast cancer are known. In some embodiments, the breast cancer can be ER positive (ER+) breast cancer. In some embodiments, the breast cancer can be ER positive, HER2-negative (ER+/HER2−) breast cancer.

In some embodiments, the breast cancer is classified by HER2 status. In some embodiments, the breast cancer is HER2-positive (HER2+) breast cancer. In some embodiments, the breast cancer is HER2-low, breast cancer.

In some embodiments, the breast cancer can be local breast cancer (as used herein, “local” breast cancer means the cancer has not spread to other areas of the body). In other embodiments, the breast cancer can be metastatic breast cancer. In still other embodiments, the breast cancer can be triple-negative breast cancer.

A subject can have a breast cancer that has not been previously treated.

In some cases, following breast cancer treatment, a subject can relapse or have reoccurrence of breast cancer. As used herein, the terms “relapse” and “reoccurrence” are used in their normal sense as understood by those skilled in the art. Thus, the breast cancer can be recurrent breast cancer. In some embodiments, the subject has relapsed after a previous treatment for breast cancer. For example, the subject has relapsed after receiving one or more treatments with a SERM, a SERD and/or aromatase inhibitor, such as those described herein.

Within ESR1, several amino acid mutations have been identified. Mutations in ESR1 have been proposed as playing a role in resistance. There are several therapies for inhibiting estrogen receptors, including selective ER modulators (SERM), selective ER degraders (SERD) and aromatase inhibitors. One issue that can arise from the aforementioned cancer therapies is the development of resistance to the cancer therapy. Acquired resistance to cancer therapy, such as endocrine therapy, has been noted in nearly one-third of women treated with tamoxifen and other endocrine therapies. See Alluri et al., “Estrogen receptor mutations and their role in breast cancer progression” Breast Cancer Research (2014) 16:494. Researchers have suspected mutations in the estrogen receptor as one of the reasons for acquired resistance to cancer therapy, such as endocrine therapy. Thus, there is a need for compounds that can treat breast cancer wherein the cancer has one or more mutations within ESR1.

Some embodiments disclosed herein are relate to the use of a combination of compounds described herein, such as Compound (A) and Compound (B), along with pharmaceutically acceptable salts of any of the foregoing, in the manufacture for a medicament for treating breast cancer in a subject in need thereof, wherein the breast cancer has at least one point mutation (such as 1, 2, 3, 4 or more than 4 point mutations) within the Estrogen Receptor 1 (ESR1) that encodes Estrogen receptor alpha (ERα). Other embodiments relate herein are directed to the use of a combination of compounds described herein that includes an effective amount of Compound (A), including pharmaceutically acceptable salts thereof, and an effective amount of Compound (B), or a pharmaceutically acceptable salt thereof, along with pharmaceutically acceptable salts thereof, for treating breast cancer in a subject in need thereof, wherein the breast cancer has at least one point mutation within the Estrogen Receptor 1 (ESR1) that encodes Estrogen receptor alpha (ERα). Still other embodiments disclosed herein are relate to a method of treating breast cancer in a subject in need thereof with a combination of compounds described herein (such as Compound (A) and Compound (B), along with pharmaceutically acceptable salts of any of the foregoing), wherein the breast cancer has at least one point mutation (for example, 1, 2, 3, 4 or more than 4 point mutations) within the Estrogen Receptor 1 (ESR1) that encodes Estrogen receptor alpha (ERα).

In some embodiments, the mutation can be in the ligand binding domain (LBD) of ESR1. In some embodiments, one or more mutations can be at an amino acid selected from: A593, S576, G557, R555, L549, A546, E542, L540, D538, Y537, L536, P535, V534, V533, N532, K531, C530, H524, E523, M522, R503, L497, K481, V478, R477, E471, S463, F461, S432, G420, V418, D411, L466, S463, L453, G442, M437, M421, M396, V392, M388, E380, G344, S338, L370, S329, K303, A283, S282, E279, G274, K252, R233, P222, G160, N156, P147, G145, F97, N69, A65, A58 and S47. In some embodiments, one or more mutations can be at an amino acid selected from: D538, Y537, L536, P535, V534, S463, V392 and E380. In some embodiments, one or more mutations can be at an amino acid selected from: D538 and Y537.

In some embodiments, one or more mutations can be selected from: K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H.

Some embodiments disclosed herein are relate to the use of a combination of compounds that includes an effective amount of Compound (A), including pharmaceutically acceptable salts thereof, and an effective amount of one or more of Compound (B), or a pharmaceutically acceptable salt thereof, in the manufacture for a medicament for treating breast cancer in a subject in need thereof, wherein the breast cancer does not include at least one point mutation (for example, a point mutation within the Estrogen Receptor 1 (ESR1) that encodes Estrogen receptor alpha (ERα)). Other embodiments relate herein are directed to the use of a combination of compounds that includes an effective amount of Compound (A) and an effective amount of one or more of Compound (B), along with pharmaceutically acceptable salts of any of the foregoing, for treating breast cancer in a subject in need thereof, wherein the breast cancer does not include has at least one point mutation, such as a point mutation within the Estrogen Receptor 1 (ESR1) that encodes Estrogen receptor alpha (ERα). Still other embodiments disclosed herein are relate to a method of treating breast cancer in a subject in need thereof with a combination of compounds described herein (for example, a combination of Compound (A) and Compound (B), or a pharmaceutically acceptable salt of any of the foregoing), wherein the breast cancer does not include has at least one point mutation within the Estrogen Receptor 1 (ESR1) that encodes Estrogen receptor alpha (ERα) (for example, a point mutation within the Estrogen Receptor 1 (ESR1) that encodes Estrogen receptor alpha (ERα)).

As provided herein, several studies have shown that a potential cause of resistance in ER-positive breast cancer is due to acquired mutations in ESR1 due to endocrine therapy. In some embodiments, the subject had been previously treated with one or more selective ER modulators. For example, subject had been treated previously with one or more selected ER modulators selected from tamoxifen, raloxifene, ospemifene, bazedoxifene, toremifene and lasofoxifene, or a pharmaceutically acceptable salt of any of the foregoing. In some embodiments, the subject had been treated previously with one or more selective ER degraders, such as fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en -1-yl)phenyl) acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(6S,8R)-8-methyl-7-(2,2,2-trifluoroethyl) -6,7,8,9-tetrahydro-3H-pyrazolo[4,3-f]isoquinolin-6-yl]pyridin-3-amine (AZD9833), OP-1250 and LY3484356, or pharmaceutically acceptable salt of any of the foregoing. In some embodiments, the subject had been treated previously with one or more aromatase inhibitors. The aromatase inhibitors can be a steroidal aromatase inhibitor or a non-steroidal aromatase inhibitor. For example, the one or more aromatase inhibitors can be selected from (exemestane (steroidal aromatase inhibitor), testolactone (steroidal aromatase inhibitor); anastazole (non-steroidal aromatase inhibitor) and letrazole (non-steroidal aromatase inhibitor), including pharmaceutically acceptable salts of any of the foregoing.

In some embodiments, the breast cancer can be present in subject, wherein the subject can be a woman. As women approach middle-age, a woman can be in a stage of menopause. In some embodiments, the subject can be a premenopausal woman. In other embodiments, the subject can be a perimenopausal woman. In still other embodiments, the subject can be a menopausal woman. In yet still other embodiments, the subject can be a postmenopausal woman. In other embodiments, the breast cancer can be present in a subject, wherein the subject can be a man. The serum estradiol level of the subject can vary. In some embodiments, the serum estradiol level (E2) of the subject can be in the range of >15 pg/mL to 350 pg/mL. In other embodiments, the serum estradiol level (E2) of the subject can be ≤15 pg/mL. In other embodiments, the serum estradiol level (E2) of the subject can be ≤10 pg/mL.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compounds (A) and/or (B), or pharmaceutically acceptable salts of any of the foregoing, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Efficacy Study Procedures

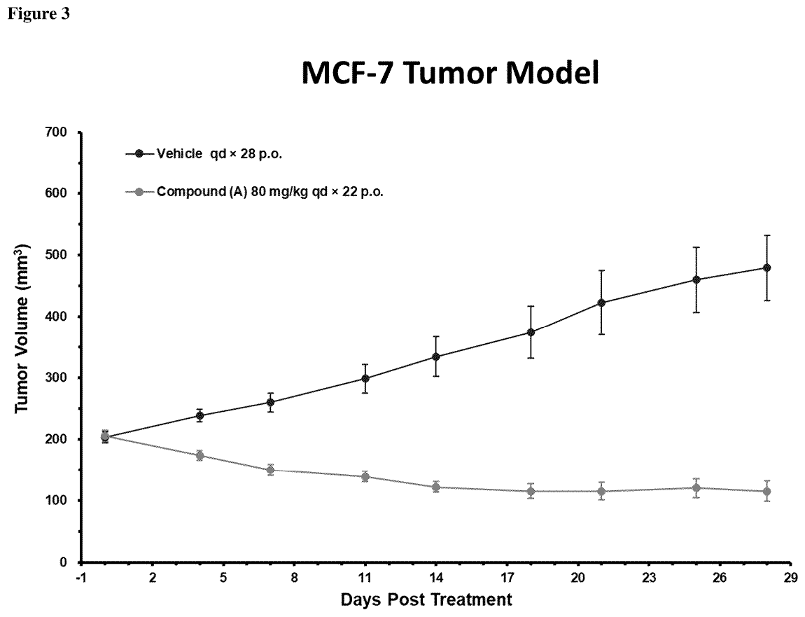

The antitumor activity of Compound (A) was assessed using the MCF-7 xenograft model. Each mouse was inoculated subcutaneously on the 2^{nd} right mammary fat pad with 1.5×10⁷ MCF-7 tumor cells in 200 μL DMEM Matrigel mixture (1:1 ratio) without serum for the tumor development. In addition, estradiol benzoate injection was delivered by s.c. (40 ug/20 uL, twice weekly). When mean tumor size reached 204 mm³, animals were randomized into 2 groups (10 animals/group) and the treatments were initiated. Compound (A) or vehicle control was administered once daily by oral gavage and continued for 28-days. Study endpoints included daily body weight, clinical observations, tumor volume. FIG. 3 showed that the dose level 80 mg/kg of Compound (A) as a single agent produced robust inhibition of tumor growth (132.6%) and tumor regression. In FIG. 3, the top line is vehicle, and the bottom line is Compound (A) (80 mg/kg qd×22 p.o). There were no adverse clinical observations in any dose group and there was no significant impact on mean body weights.

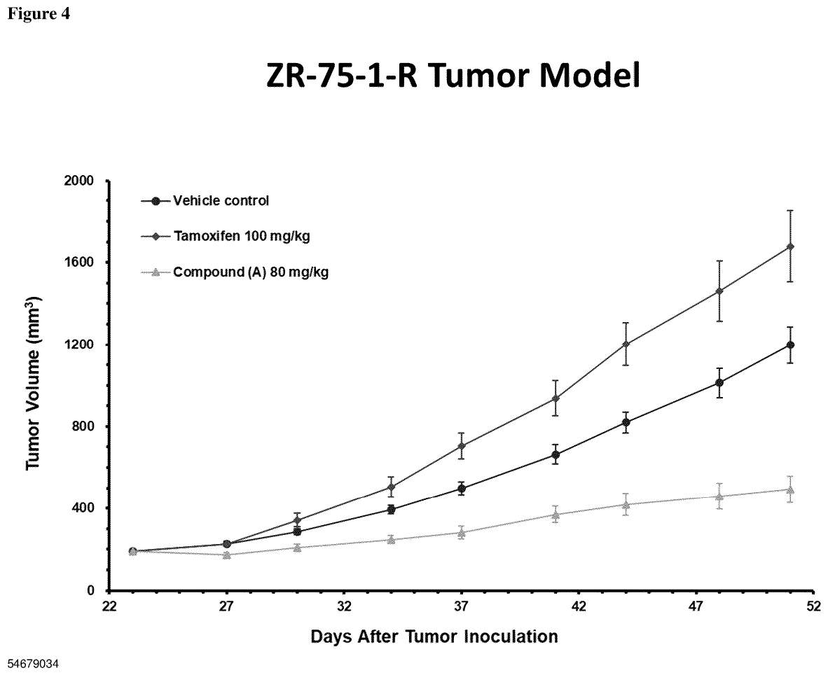

Compound (A)'s efficacy as a single agent was evaluated in ZR-75-1 tamoxifen resistant tumor model. The tamoxifen resistant ZR-75-1 tumor cells (ZR-75-1R) were maintained in vitro as monolayer culture in RPMI1640 Medium supplemented with 10% fetal bovine serum and 10 μM tamoxifen at 37° C. in an atmosphere of 5% CO2 incubator. Each mouse was then inoculated subcutaneously on the right flank with 1×10⁷ ZR-75-1R tumor cells in 100 μL RPMI-1640 Matrigel mixture (1:1 ratio) without serum for the tumor development. In addition, estradiol benzoate injection was delivered by s.c. (40 ug/20 uL, twice weekly). Mice were then randomized into 3 groups when mean tumor size reached 191 mm³ and treatment was started. Tamoxifen was dosed at 100 mg/kg 5 days per week for three weeks, Compound (A) was orally dosed at 80 mg/kg daily for 28 days. Study endpoints included daily body weight, clinical observations, tumor volume. In FIG. 4, the top line is Tamoxifen (100 mg/kg), the middle line is vehicle, and the bottom line is Compound (A) (80 mg/kg). As shown in FIG. 4, Tamoxifen induced −47.9% tumor growth inhibition post 28 days treatment and indicated this is tamoxifen resistant breast cancer model. Compound (A) at 80 mg/kg produced strong antitumor activities with 69.9% TGI (tumor growth inhibition). No other gross clinical abnormalities were observed during the treatment period and animal body loss was manageable by giving animals dosing holiday.

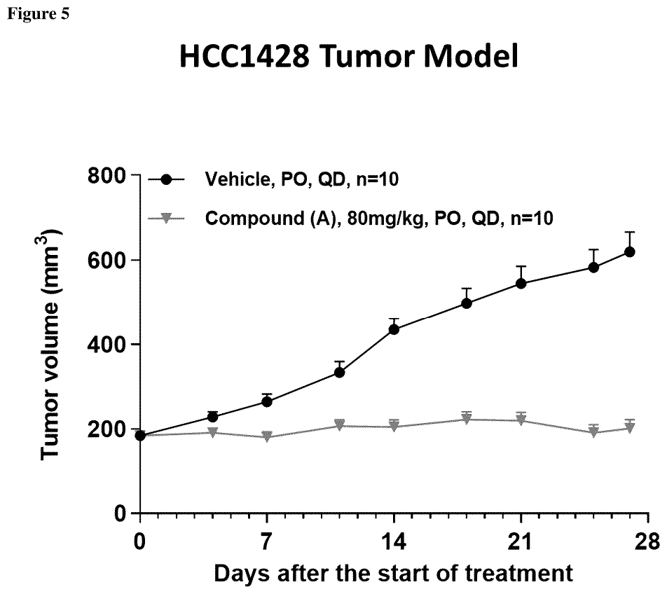

The activity of Compound (A) as evaluated in mice bearing human HCC1428 breast cancer xenograft tumors. Each mouse was inoculated subcutaneously at the right flank with HCC1428 tumor cells (1×10⁷) in 0.2 mL mixture of PBS and Matrigel (PBS: Matrigel=1:1) for tumor development, which has been subcutaneously implanted with 17beta-estradiol tablets (0.18 mg, 90-day release) two days before cell implantation. Treatments were started on day 21 after tumor inoculation when the average tumor size reached approximately 185 mm^{3.} The animals were assigned into two groups using an excel-based randomization software performing stratified randomization based upon their tumor volumes. Each group consisted of 10 tumor bearing mice. The vehicle control or Compound (A) at 80 mg/kg was administrated once daily to the mice by oral gavage for 28 days. In FIG. 5, the top line is vehicle, and the bottom line is Compound (A). As shown in FIG. 5, the treatment with Compound (A) as a single agent produced a significant inhibition of HCC1428 tumor growth relative to the vehicle group with 96.2% tumor growth inhibition (TGI) observed at 80 mg/kg of Compound (A). There were no adverse clinical observations and there was no significant body weight loss observed in the Compound (A) treated animals.

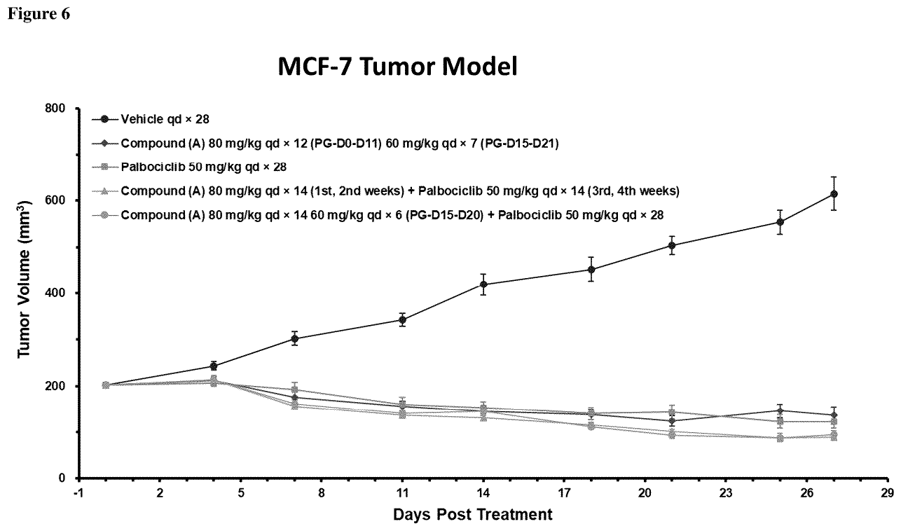

The antitumor activity of Compound (A) in combination with a CDK4/6 inhibitor, palbociclib was evaluated in the MCF-7 xenograft model. Each mouse was inoculated subcutaneously on the right flank with the MCF-7 tumor cells (1.5×10⁷) in 200 μL DMEM Matrigel mixture (1:1 ratio) without serum for the tumor development. In addition, estradiol benzoate injection was delivered by s.c. (40 ug/20 uL, twice weekly). When mean tumor size reached 202 mm³, mice were randomized into 5 groups (10 animals/group) and treatment was started. Animals were dosed with vehicle, 80/60 mg/kg Compound (A) alone, 50 mg/kg palbociclib alone, 80 mg/kg Compound (A) in combination with 50 mg/kg Palbociclib by sequential dosing schedule (alternated per two weeks) or Compound (A) at 80/60 mg/kg in combination with palbociclib at 50 mg/kg. Treatment periods were 28 days. In FIG. 6, the top line (indicated with circles) is vehicle. As shown in FIG. 6, Compound (A) at 80/60 mg/kg or palbociclib at 50 mg/kg alone gave around 116% and 119% TGI, respectively. The sequential treatment with Compound (A) and palbociclib induced 127% TGI; thus, this data shows better antitumor activity than monotherapy with either Compound (A) or palbociclib, respectively. Co-treatment of Compound (A) at 80/60 mg/kg and palbociclib at 50 mg/kg also induced deeper antitumor activity with 126.2% TGI. Furthermore, Compound (A) in combination with palbociclib induced around 55% tumor regression. By comparison, Compound (A) or palbociclib alone treatment only induced 32.7% and 39.1% tumor regression, respectively. Overall, Compound (A) in combination with Palbociclib significantly improved the efficacy.

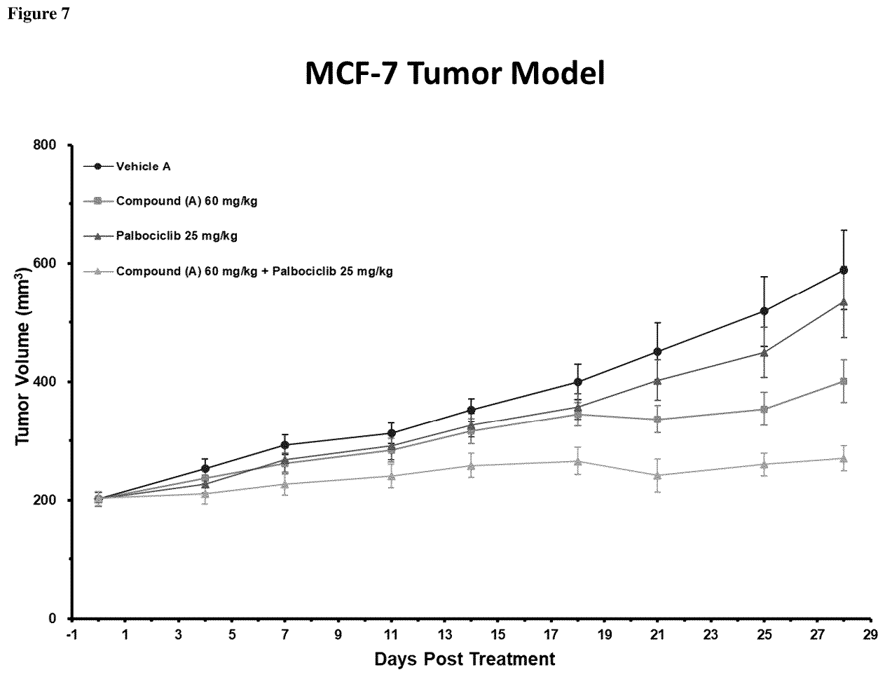

The antitumor activity of Compound (A) in combination with a CDK4/6 inhibitor, palbociclib at a lower dose was evaluated in the MCF-7 xenograft model. Each mouse was inoculated subcutaneously on the 2^{nd} right mammary fat pad with 1.5×10⁷ MCF-7 cells in 200 μL DMEM Matrigel mixture (1:1 ratio) without serum for the tumor development. In addition, estradiol benzoate injection was delivered by s.c. (40 ug/20 uL, twice weekly). The treatments were started when mean tumor size reached 203 mm³. Mice were randomized into 4 groups (8 mice per group) and treated with vehicle control, 60 mg/kg Compound (A) alone, 25 mg/kg Palbociclib alone, Compound (A) at 60 mg/kg in combination with 25 mg/kg palbociclib. Study endpoints included daily body weight, clinical observations, tumor volume. As shown in FIG. 7, Compound (A) at 60 mg/kg alone (second from the bottom line) and palbociclib at 25 mg/kg alone (second from the top line indicated with triangles) induced 48.6% TGI and 14.2% TGI respectively, Compound (A) at 60 mg/kg in the combination with palbociclib at 25 mg/kg (bottom line indicated with triangles) resulted in 82.7% TGI. Thus, Compound (A) in combination with palbociclib significantly improved the efficacy. All animals were well tolerated for the treatment.

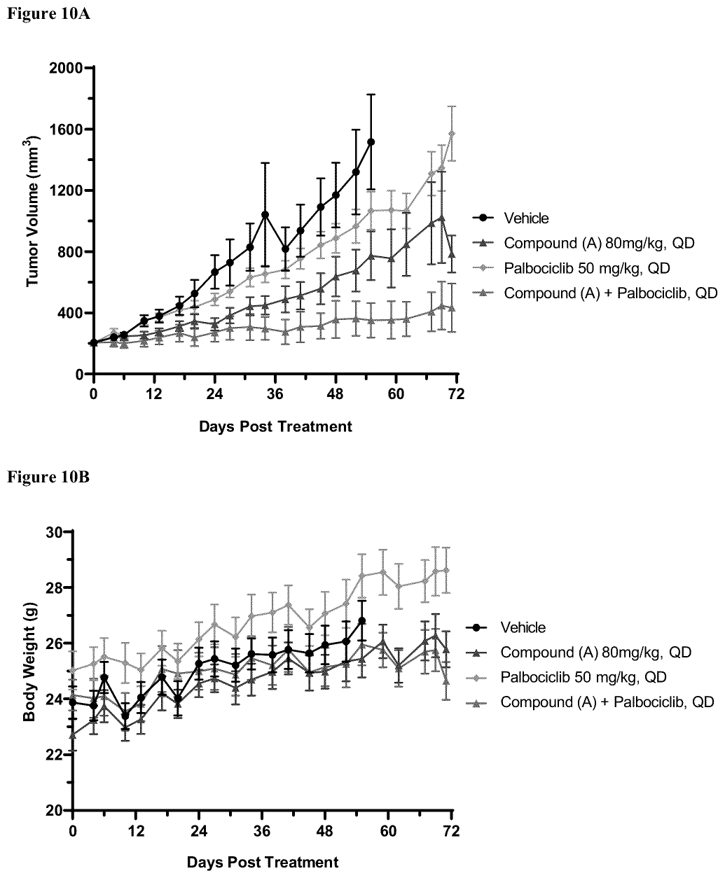

The antitumor activity of Compound (A) in combination with palbociclib was further evaluated in a palbociclib-resistant breast cancer PDX model (CTG-1207). Mice were inoculated subcutaneously with palbociclib-resistant breast cancer cells in DMEM Matrigel mixture (1:1 ratio) without serum for tumor development. Once tumor size reached approximately 200 mm³, mice were randomized into 4 groups (8 mice per group) and treated with vehicle control, 80 mg/kg Compound (A) alone, 50 mg/kg Palbociclib alone, or Compound (A) at 80 mg/kg in combination with 50 mg/kg palbociclib. Study endpoints included daily body weight, clinical observations, tumor volume. As shown in FIG. 10A, Compound (A) at 80 mg/kg alone (second from the bottom line indicated with triangles) reduced tumor growth while palbociclib at 50 mg/kg alone (second from the top line indicated with diamonds) demonstrated lower TGI. Compound (A) at 80 mg/kg in combination with palbociclib at 50 mg/kg (bottom line indicated with triangles) resulted in enhanced TGI in palbociclib-resistant PDX breast cancer models. Thus, Compound (A) in combination with palbociclib significantly improved the efficacy. All animals were well tolerated for the treatment as demonstrated by FIG. 10B.

### Study 1-Part 1

The HER2+ JIMT-1 breast cancer cell line was used for tumor xenograft inoculations and subsequent treatment. The JIMT-1 breast cancer cell line has been described in literature to be relatively resistant to HER2-targeted therapy (JIMT-1 cell line was established from pleural metastasis of a 62-year-old patient with breast cancer who was clinically resistant to trastuzumab (See Tanner et al. Mol Cancer Ther (2004) 3(12):1585-1592)), including to weekly administration of trastuzumab. To test if Compound (A) can improve the anti-tumor effects of HER2-targeted treatment, NOD/SCID mice were inoculated subcutaneously on the right flank with the single cell suspension of 95% viable tumor cells (5×10⁶) in 100 μL DMEM Matrigel mixture (1:1 ratio) for tumor development. Treatment was started when mean tumor size reached 204 mm³. Mice were then randomized into 5 groups and treatments were administered to the tumor-bearing mice accordingly to the study design in Table 2.

TABLE 2

| | | Animals | Dose | Vol | | |
|---|---|---|---|---|---|---|
| Groups | Treatment | per group | (mg/kg) | (μL/g) | Route | Frequency |
| 1 | Vehicle A | 10 | — | 10 | p.o. | qd × 28 |
| 2 | Vehicle B | 10 | — | 10 | i.p. | qw × 4 |
| 3 | Compound (A) | 10 | 60 | 10 | p.o. | qd × 28 |
| 4 | Trastuzumab | 10 | 10 | 10 | i.p. | qw × 4 |
| 5 | Compound (A) + | 10 | 60 + | 10 + | p.o. + | qd × 28 + |
| | Trastuzumab | | 10 | 10 | i.p. | qw x 4 |

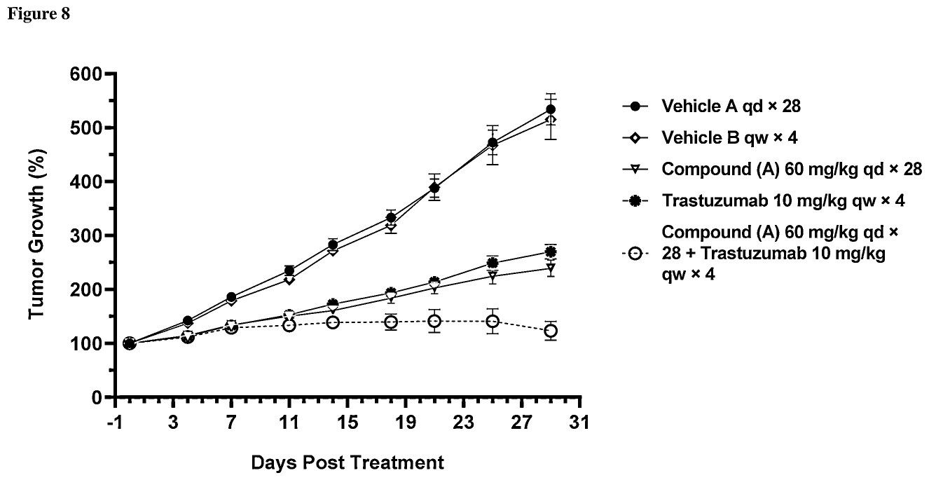

Treatment with Compound (A) (60 mg/kg) alone or trastuzumab (10 mg/kg) alone resulted in tumor growth inhibition (TGI) values on Day 29 of 68% and 59%, respectively, compared to their respective vehicle controls (administered either p.o. or i.p., respectively). In FIG. 8, the top line (indicated with circles) is Vehicle A, and the third from the bottom line (indicated with circles) is trastuzumab (10 mg/kg) alone. The combination of Compound (A) with trastuzumab resulted in 94% TGI compared to either vehicle control on Day 29. The tumor growth curves are summarized in FIG. 8 and summarized in Table 3. These data showed that the combination of Compound (A) with trastuzumab had superior anti-tumor effects compared to either treatment as mono-therapy. All treatments, including Compound (A) alone, Trastuzumab alone, or the combination of Compound (A) with trastuzumab, were well tolerated. Table 3 summarizes the results of the experiment depicted in FIG. 8. ^{a}TGI=Tumor growth inhibition, calculated as TGI=(1-(T_{d}−T₀)/(C_{d}−-C₀))×100%, T_{d} and C_{d} were the mean tumor volumes of the treated and control animals, and T₀ and C₀ were the mean tumor volumes of the treated and control animals at the start of the experiment; ^{b.} calculated vs. Vehicle Control by Dunnett T3 Test.

TABLE 3

| | TGIª | TGIª | P | P |
|---|---|---|---|---|
| | (% ) vs. | (% ) vs. | value^{b} vs. | value^{b} vs. |
| Treatment | Vehicle A | Vehicle B | Vehicle A | Vehicle B |
| Compound (A) | 67.7 | — | <0.001 | |
| Trastuzumab | — | 59.2 | — | <0.001 |
| Compound | 94.1 | 93.9 | <0.001 | <0.001 |
| (A) + | | | | |
| Trastuzumab | | | | |

### Study 1-Part 2

Next, mice in the two vehicle groups were randomized on Day 29 to be treated with either vehicle, Compound (A) alone, trastuzumab alone or the combination of Compound (A) with trastuzumab according to Table 4. At this point, tumors had reached approximately 1000 mm³ and therefore, treatment of these mice occurred at high initial tumor burden.

TABLE 4

| | | Animals | Dose | Vol | | |
|---|---|---|---|---|---|---|
| Groups | Treatment | per group | (mg/kg) | (μL/g) | Route | Frequency |
| 6 | Vehicle | 2 | — | — | p.o. | qd × 15 |
| 7 | Compound (A) | 6 | 60 | 10 | p.o. | qd × 36 |
| 8 | Trastuzumab | 6 | 10 | 10 | i.p. | qw × 6 |
| 9 | Compound (A) + | 6 | 60 + | 10 + | p.o. + | qd × 36 + |
| | Trastuzumab | | 10 | 10 | i.p. | qw x 6 |

### Study 2

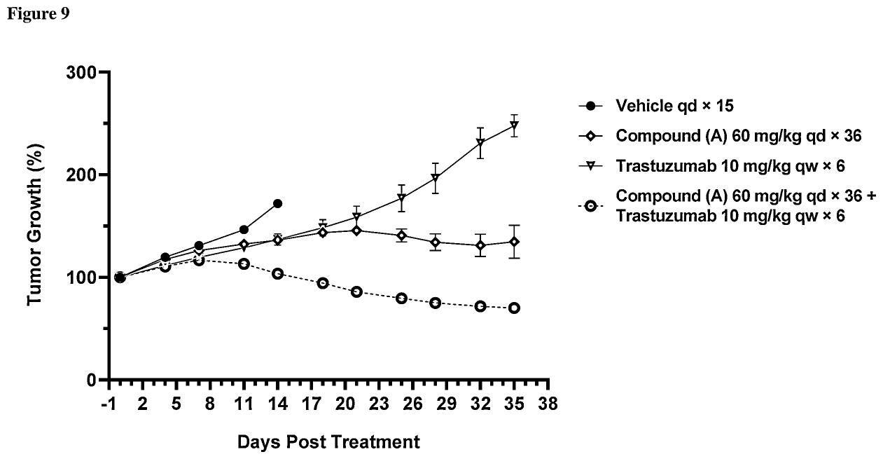

Treatment with Compound (A) (60 mg/kg) alone or trastuzumab (10 mg/kg) alone on Day 14 of the second part of the experiment resulted in TGI values of 68.2% and 68.0%, respectively, compared to vehicle controls. The combination of Compound (A) with trastuzumab resulted in 97% TGI compared to vehicle control on Day 14 of second part of the experiment (FIG. 9 and Table 5). Table 5 summarizes the results (tumor growth inhibition) of the experiment depicted in FIG. 9. ^{a}TGI=Tumor growth inhibition on Day 14. On Day 35 of the second part of the experiment, the combination of Compound (A) with trastuzumab had resulted in 30.6% tumor regression (FIG. 9 and Table 6). Table 6 summarizes the results (tumor regression) of the experiment depicted in FIG. 9. ^{a}Tumor Regression=(1−(T_{d}/T₀))×100%.

TABLE 5

| Treatment | TGIª (%) on Day 14 |
|---|---|
| Compound (A) | 68.2 |
| Trastuzumab | 68.0 |
| Compound | 97.4 |
| (A) + | |
| Trastuzumab | |

TABLE 6

| | Tumor Regression^{a} |
|---|---|
| Treatment | (%) on Day 35 |
| Compound (A) | — |
| Trastuzumab | — |
| Compound | 30.6 |
| (A) + | |
| Trastuzumab | |

These data showed that the combination of Compound (A) with trastuzumab showed significantly better anti-tumor effects compared to either treatment as mono-therapy, when mice were treated at a high initial tumor burden. Moreover, all treatment regimens were tolerated well.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. Use of a combination of compounds for treating a disease or condition, wherein the combination includes an effective amount of Compound (A) and an effective amount of Compound (B), or a pharmaceutically acceptable salt of any of the foregoing, wherein:
Compound (A) is 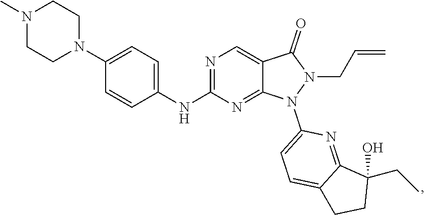 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof; and
Compound (B) is selected from the group consisting of a CDK4/6 inhibitor, a HER-2 small molecule inhibitor, a HER-2 antibody, a HER-2 antibody-drug conjugate and a HER2 bispecific antibody, or a pharmaceutically acceptable salt of any of the foregoing;
wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002 and MM-D37K, or a pharmaceutically acceptable salt of any of the foregoing;
wherein the HER-2 antibody is selected from the group consisting of trastuzumab, trastuzumab-dkst, pertuzumab and ZW25, or a pharmaceutically acceptable salt of any of the foregoing;
wherein the HER-2 antibody-drug conjugate is selected from the group consisting of Ado-trastuzumab emtansine (T-DM1), ARX788, ALT-P7, DS8201a, MEDI4276, MM302, PF-06804103, SYD985, XMT-1522, ZW49, MRG002, GQ1001, A166, RC48-ADC, BDC-1001, and FS-1502 or a pharmaceutically acceptable salt of any of the foregoing;
wherein the HER2 bispecific antibody is selected from the group consisting of margetuximab, ertumaxomab, HER2Bi-aATC, MM-111, MCLA-128, BTRC4017A, GBR-1302 and PRS-343, or a pharmaceutically acceptable salt of any of the foregoing; and
wherein the HER-2 small molecule inhibitor is selected from the group consisting of tucatinib, lapatinib and neratinib, or a pharmaceutically acceptable salt of any of the foregoing.

2. The use of claim 1, wherein Compound (B) is a CDK4/6 inhibitor, or a pharmaceutically acceptable salt thereof.

3. The use of claim 2, wherein the CDK4/6 inhibitor is palbociclib.

4. The use of claim 2, wherein the CDK4/6 inhibitor is abemaciclib.

5. The use of claim 2, wherein the CDK4/6 inhibitor is ribociclib.

6. The use of claim 2, wherein the CDK4/6 inhibitor is trilaciclib.

7. The use of claim 1, wherein Compound (B) is an HER-2 antibody, or a pharmaceutically acceptable salt thereof.

8. The use of claim 7, wherein the HER-2 antibody is trastuzumab.

9. The use of claim 1, wherein Compound (B) is a HER-2 antibody-drug conjugate, or a pharmaceutically acceptable salt thereof.

10. The use of claim 1, wherein Compound (B) is a HER2 bispecific antibody, or a pharmaceutically acceptable salt thereof.

11. The use of any one of claims 1-10, wherein the disease or condition is a cancer selected from the group consisting of lung cancer, gastric cancer, gastroesophageal junction adenocarcinoma and breast cancer.

12. The use of claim 11, wherein the breast cancer is selected from the group consisting of triple-negative breast cancer, ER+ breast cancer, HER2-positive (HER2 +) breast cancer and HER2-low breast cancer.

13. Use of an effective amount of Compound (A), or a pharmaceutically acceptable salt of any of the foregoing, in the preparation of a medicament for treating ER+ breast cancer, wherein
Compound (A) is 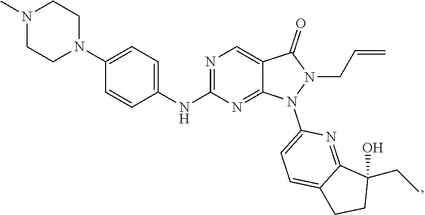 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof.

14. The use of any one of claims 11-13, wherein the breast cancer that does not include any point mutations ER mutations.

15. The use of any one of claims 11-13, wherein the breast cancer has at least one point mutation within the Estrogen Receptor 1 (ESR1) that encodes Estrogen receptor alpha (ERα), wherein the mutation is selected from the group consisting of: K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V3921, V418E, V478L, V533M, V534E, Y537D and Y537H.

16. The use of any one of claims 11-13, wherein the cancer is ER positive breast cancer.

17. The use of any one of claims 11-13, wherein the cancer is ER positive/HER2-negative breast cancer.

18. The use of any one of claims 11-13, wherein the cancer is local breast cancer.

19. The use of any one of claims 11-13, wherein the cancer is metastatic breast cancer.

20. The use of any one of claims 11-13, wherein the cancer is recurrent breast cancer.

21. The use of any one of claims 11-20, wherein the cancer is breast cancer that has been previously treated with an endocrine therapy.

22. The use of claim 21, wherein the treatment was with a selective ER modulator (SERM).

23. The use of claim 22, wherein the selective ER modulator is selected from the group consisting of tamoxifen, raloxifene, ospemifene, bazedoxifene, toremifene and lasofoxifene, or a pharmaceutically acceptable salt of any of the foregoing. 24 The use of claim 21, wherein the treatment was with a selective ER degrader (SERD).

25. The use of claim 24, wherein the selective ER degrader is selected from the group consisting of fulvestrant, (E)-3-[3,5-Difluoro-4-[(1R,3R)-2-(2-fluoro-2-methylpropyl)-3-methyl -1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo [7]annulene-3-carboxylic acid (SAR439859), N-[1-(3-fluoropropyl)azetidin-3-yl]-6-[(6S,8R)-8-methyl-7-(2,2,2-trifluoroethyl) -6,7,8,9-tetrahydro-3H-pyrazolo[4,3-f]isoquinolin-6-yl]pyridin-3-amine (AZD9833), OP-1250 and LY3484356, or a pharmaceutically acceptable salt of any of the foregoing.

26. The use of claim 21, wherein the treatment was with an aromatase inhibitor.

27. The use of claim 26, wherein the aromatase inhibitor is a steroidal aromatase inhibitor.

28. The use of claim 27, wherein the steroidal aromatase inhibitor is selected from the group consisting of exemestane and testolactone, or a pharmaceutically acceptable salt of any of the foregoing.

29. The use of claim 26, wherein the aromatase inhibitor is a non-steroidal aromatase inhibitor.

30. The use of claim 29, wherein the non-steroidal aromatase inhibitor is selected from the group consisting of anastazole and letrazole, or a pharmaceutically acceptable salt of any of the foregoing.

31. The use of any one of claims 11-20, wherein the breast cancer has not been previously treated.

32. The use of any one of claim 11-31, wherein the breast cancer is present in a woman.

33. The use of claim 32, wherein the woman is a premenopausal woman.

34. The use of claim 32, wherein the woman is a perimenopausal woman.

35. The use of claim 32, wherein the woman is a menopausal woman.

36. The use of claim 32, wherein the breast cancer is present in a postmenopausal woman.

37. The use of any one of claims 11-31, wherein the breast cancer is present in a man.

38. The use of any one of claims 11-37, wherein the breast cancer is present in a subject that has a serum estradiol level in the range of >15 pg/mL to 350 pg/mL. 39 The use of any one of claims 11-37, wherein the breast cancer is present in a subject that has a serum estradiol level ≤15 pg/mL.

40. The use of any one of claims 11-37, wherein the breast cancer is present in a subject that has a serum estradiol level ≤10 pg/mL.

41. A method of treating cancer comprising administering to a subject an effective amount of Compound (A) and an effective amount of Compound (B), or a pharmaceutically acceptable salt of any of the foregoing, wherein:
Compound (A) is 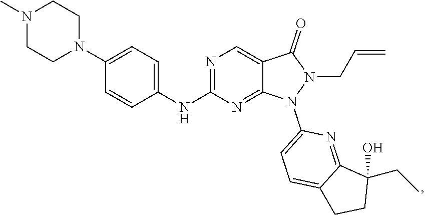 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof; and
Compound (B) is selected from the group consisting of a CDK4/6 inhibitor, a HER-2 small molecule inhibitor, a HER-2 antibody, a HER-2 antibody-drug conjugate and a HER2 bispecific antibody, or a pharmaceutically acceptable salt of any of the foregoing;
wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002 and MM-D37K, or a pharmaceutically acceptable salt of any of the foregoing;
wherein the HER-2 antibody is selected from the group consisting of trastuzumab, trastuzumab-dkst, pertuzumab and ZW25, or a pharmaceutically acceptable salt of any of the foregoing;
wherein the HER-2 antibody-drug conjugate is selected from the group consisting of Ado-trastuzumab emtansine (T-DM1), ARX788, ALT-P7, DS8201a, MEDI4276, MM302, PF-06804103, SYD985, XMT-1522, ZW49, MRG002, GQ1001, A166, RC48-ADC, BDC-1001, and FS-1502, or a pharmaceutically acceptable salt of any of the foregoing;
wherein the HER2 bispecific antibody is selected from the group consisting of margetuximab, ertumaxomab, HER2Bi-aATC, MM-111, MCLA-128, BTRC4017A, GBR-1302 and PRS-343, or a pharmaceutically acceptable salt of any of the foregoing; and
wherein the HER-2 small molecule inhibitor is selected from the group consisting of tucatinib, lapatinib, and neratinib, or a pharmaceutically acceptable salt of any of the foregoing.

42. The method of claim 41, wherein Compound (B) is a CDK4/6 inhibitor, or a pharmaceutically acceptable salt thereof.

43. The method of claim 42, wherein the CDK4/6 inhibitor is palbociclib.

44. The method of claim 42, wherein the CDK4/6 inhibitor is abemaciclib.

45. The method of claim 42, wherein the CDK4/6 inhibitor is ribociclib.

46. The method of claim 42, wherein the CDK4/6 inhibitor is trilaciclib.

47. The method of claim 42, wherein Compound (B) is an HER-2 antibody, or a pharmaceutically acceptable salt thereof.

48. The method of claim 42, wherein the HER-2 antibody is trastuzumab.

49. The method of claim 42, wherein Compound (B) is a HER-2 antibody-drug conjugate, or a pharmaceutically acceptable salt thereof.

50. The method of claim 42, wherein Compound (B) is a HER-2 antibody-drug conjugate, or a pharmaceutically acceptable salt thereof, wherein the HER-2 antibody is trastuzumab.

51. The method of claim 42, wherein Compound (B) is fam-trastuzumab-deruxtecan-nxki (DS8201a).

52. The method of claim 42, wherein Compound (B) is a HER2 bispecific antibody, or a pharmaceutically acceptable salt thereof.

53. The method of any one of claims 41-52, wherein the cancer is selected from the group consisting of lung cancer, gastric cancer, gastroesophageal junction adenocarcinoma and breast cancer.

54. The method of claim 53, wherein the cancer is breast cancer selected from the group consisting of triple-negative breast cancer, ER+ breast cancer, HER2-positive (HER2 +) breast cancer and HER2-low breast cancer.

55. A method of treating triple negative breast cancer comprising administering to a subject an effective amount of Compound (A) and an effective amount of Compound (B), or a pharmaceutically acceptable salt of any of the foregoing, wherein:
Compound (A) is 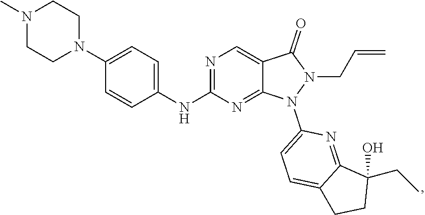 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof; and
Compound (B) is fam-trastuzumab-deruxtecan-nxki (DS8201a).
